(19) United States
(12) Patent Application Publication
(10) Pub. No.: US 2009/0238773 A1
(43) Pub. Date: Sep. 24, 2009

(54) Use of Morpholino Compounds for the Treatment of Halitosis

(76) Inventor: Rolf Valter Attstrom, Brainz (CH)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/092,933

(22) PCT Filed: Jan. 5, 2007
(86) PCT No.: PCT/GB2007/000025

§ 371 Date: Aug. 8, 2008

(30) Foreign Application Priority Data
Jan. 10, 2006 (GB) 0600431.1

(51) Int. Cl. A61K 8/49; A61K 31/535; C07D 295/00

(52) U.S. Cl. 424/48; 514/238.8; 424/49; 544/170

(57) ABSTRACT

Use of a morpholino compound having the general formula (I) wherein R₁ is a straight or branched alkyl group containing 8 to 16 carbon atoms at the 2- or 3-position of the morpholino ring, and R₂ is a straight or branched alkyl group containing 2 to 10 carbon atoms, substituted with a hydroxy group except in the alpha-position, or a pharmaceutically acceptable salt thereof, in the manufacture of a medicament for the prevention or treatment of halitosis.

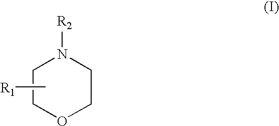

### FIELD OF THE INVENTION

The present invention relates to the prevention and treatment of halitosis.

### BACKGROUND OF THE INVENTION

Halitosis is commonly known as “bad breath” and has been defined as an unpleasant or offensive odour emanating from the oral cavity (Roldan et al, J Periodontol. 2005 Jun; 76(6): 1025-33). Although the cause of halitosis can be non-oral, approximately 90% of all offensive odours originate in the mouth (Delanghe et al, Quintessence Int. 1999 May; 30(5): 307-10). More specifically, approximately 40% of all malodour is thought to originate from the dorso-posterior region of the tongue (Delanghe et al, supra).

The principal components of “bad breath” are Volatile Sulphur Compounds (VSCs), especially hydrogen sulphide, methyl mercaptan and dimethylsuphide, or compounds such as putrescine, cadaverine, butyric and propionic acids. These compounds result from proteolytic degradation of various sulphur-containing substrates in food debris, saliva, blood and epithelial cells, by predominantly anaerobic Gram-negative microorganisms in the oral cavity. Substrates for VSC production include the sulphur-containing amino acids methionine, cysteine and cystine, present in saliva or crevicular fluids (Tonzetich J, J Periodontol. 1977 Jan; 48(1): 13-20; Persson et al, Oral Microbiol Immunol. 1990 Aug; 5(4): 195-201). Bacteria such as P. gingivalis, Actinobacillus actinomycetemcomitans, Fusobacterium nucleatum, Tannerella forsythensis, Prevotella intermedia, Campylobacter rectus, Eubacterium sp and Spirochetes are known to produce VSCs (Tonzetich J, supra). Oral infections such as gingivitis and periodontitis have been suggested as possible causes of halitosis (Yaegaki K. and Coil J M., J Can Dent Assoc. 2000 May; 66(5): 257-61).

Effective management of halitosis includes the use of antimicrobial mouthwashes, oral prophylaxis, professional cleaning and the treatment of oral infections and diseases. Although these methods are widespread in clinical dentistry, there are very few scientific evaluations to verify the effectiveness of the different methods. However, it is accepted that successful treatment of halitosis is associated with the reduction of production of VSCs by bacteria that are capable of utilising sulphur containing substrates, in particular the Gram-negative anaerobes. Quirynen et al (J Periodontol. 2005 May; 76(5): 705-12) concluded that VSC levels correlate to anaerobic bacteria counts in the saliva.

Roldan et al (supra) describes a study to determine the effectiveness of a combined therapeutic approach to managing halitosis. A combination of chlorhexidine and cetylpyridium chloride with zinc lactate was adminstered to subjects presenting halitosis. Total bacterial counts in the subgingiva, saliva and the tongue coating were reduced during the treatment period. In particular, a decrease in the levels and proportions of Porphyromonas gingivalis in subgingival plaque, unstimulated saliva and tongue coating was seen. This reduction was closely related to reductions in organoleptic scores, volatile sulphur compound (VSC) levels and the Winkel tongue coating index. The ratio of anaerobic to aerobic bacteria in the tongue coating also decreased during the study. The main adverse effect of the treatment was staining of the teeth.

Fine et al (J Clin Periodontol. 2005 Apr; 32(4): 335-40) reported that an essential oil mouthwash containing zinc chloride (Tartar Control Listerine® Antiseptic) reduced mean bacterial counts from the dorsum of the tongue and supragingival plaque. The reduction in bacterial load, particularly anaerobic bacteria (particularly Gram-negative anaerobes) and VSC-producing bacteria, was found to be responsible for a reduction in malodour.

There is a need for further products and techniques for effectively preventing, treating and managing halitosis.

### SUMMARY OF THE INVENTION

The present invention is based on the surprising discovery that Delmopinol (3-(4-propyl-heptyl)-4-(2-hydroxyethyl)morpholine), and derivatives thereof, is useful in preventing and treating halitosis. Although Delmopinol (and derivatives thereof is known to be useful for the removal or inhibition of dental plaque formation, as disclosed in U.S. Pat. No. 4,894,221, it has previously been demonstrated that a delmopinol mouthwash does not significantly affect the numbers or proportions of oral bacteria (Elworthy et al, J Clin Periodontol. 1995 Jul; 22(7): 527-32). As delmopinol is not known to reduce the oral bacterial load, let alone the load of Gram-negative anaerobes, it is surprising that levels of VSCs decrease following treatment with delmopinol.

According to a first aspect of the invention, a morpholino compound having the general formula (I)

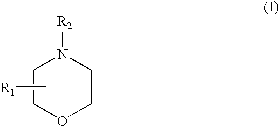

wherein R₁ is a straight or branched alkyl group containing 8 to 16 carbon atoms at the 2- or 3-position of the morpholino ring, and R₂ is a straight or branched alkyl group containing 2 to 10 carbon atoms, substituted with a hydroxy group except in the alpha-position, or a pharmaceutically acceptable salt thereof, is used in the manufacture of a medicament for the prevention or treatment of halitosis.

According to a second aspect of the invention, a kit for treating halitosis comprises a compound having the general formula (I), or a medicament containing a compound of formula (I), and instructions that the compound or medicament is to be used for the treatment of halitosis.

### DETAILED DESCRIPTION OF THE INVENTION

A morpholino compound of formula (I) can be used to treat and prevent halitosis. A morpholino compound according to the invention has the general formula (I)

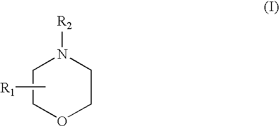

wherein R₁ is a straight or branched alkyl group containing 8 to 16 carbon atoms at the 2- or 3-position of the morpholino ring, and R₂ is a straight or branched alkyl group containing 2 to 10 carbon atoms, substituted with a hydroxy group except in the alpha-position, or pharmaceutically acceptable salts thereof. In a preferred embodiment, the sum of the carbon atoms in the groups R₁ and R₂ of the morpholino compound is at least 10, preferably between 10 and 20. In a further preferred embodiment, the R₂group terminates with the hydroxy group.

The claimed morpholino compounds are known per se and can be manufactured by any known method, for example that disclosed in U.S. Pat. No. 5,082,653 and WO90/14342, which are incorporated herein by reference.

The preferred morpholino compound for use in the invention is 3-(4-propyl-heptyl)-4-(2-hydroxyethyl)morpholine, which is commonly known as Delmopinol (CAS No. 79874-76-3).

The morpholino compounds can be used in their free base form or as a pharmaceutically acceptable salt thereof. Examples of pharmaceutically acceptable salts are the salts of acids such as acetic acid, phosphoric acid, boric acid, hydrochloric acid, maleic acid, benzoic acid, citric acid, malic acid, oxalic acid, tartaric acid, succinic acid, glutaric acid, gentisic acid, valeric acid, gallic acid, beta-resorcyclic acid, acetyl salicylic acid, salicylic acid, perchloric acid, barbituric acid, sulfanilic acid, phytic acid, p-nitro benzoic acid, stearic acid, palmitic acid, oleic acid, myristic acid, lauric acid and the like. The most preferred salts are those of hydrochloric acid. A preferred compound is delmopinol hydrochloride (CAS No. 98092-92-3).

As used herein, the term “halitosis” refers to the commonly recognised meaning of the term, i.e. “bad breath”. This may be defined as breath that has an odour that is unpleasant or offensive to the person exhaling the breath, or to others. In a preferred embodiment, the breath contains Volatile Sulphur Compounds (VSC's), including but not limited to hydrogen sulphide, methyl mercaptan or dimethylsuphide, or compounds such as putrescine, cadaverine, butyric and propionic acids. These compounds result from proteolytic degradation of various sulphur-containing substrates in food debris, saliva, blood and epithelial cells, by predominantly anaerobic Gram-negative microorganisms in the oral cavity.

As used herein, the term “treatment” refers to the reduction of halitosis in a patient, i.e. the improvement of the breath odour. Changes in breath odour can be detected by any technique. Preferred techniques include organoleptic detection or, when the breath contains VSCs, the use of a sulfide monitor. Both techniques are well-known in the art, as described in Roldan et al, supra.

The current invention is applicable equally in the fields of human and animal medicine, i.e. veterinary applications are within the scope of the invention. In the veterinary embodiment, treatment of pets including cats and dogs, and treatment of farm animals including cattle and swine, are preferred embodiments.

The compound of formula (I) may be brought into contact with the oral cavity in a conventional way, in any suitable form or amount that achieves the desired effect, i.e. reduction of halitosis. Preferably, the compound of formula (I) is in the form of a mouthwash, toothpaste, gel, dentifrice, gum or other similar preparation that will be apparent to the skilled person. The preparation can contain at least one pharmaceutically acceptable excipient. Most preferably, the compound is in the form of an aqueous mouthwash. This can be applied to the oral cavity by the patient, without the need for medical supervision. Preferably, the mouthwash is held in the mouth for at least 5 seconds, preferably greater than 10 seconds, for example one minute or more.

In a preferred embodiment, mechanical agitation, preferably brushing or scraping of the tongue, is performed simultaneously with or shortly, preferably immediately, before or after contacting the oral cavity with a compound of formula (I). Most preferably, the compound of formula (I) is applied as an aqueous mouthwash at the start of any regular (e.g. daily) oral health routine, such as before brushing the teeth and tongue.

The compound of formula (I) could be added to an existing toothpaste, gum, gel or mouthwash formulation. The compound of formula (I) may be added in combination with at least one anti-microbial, preferably anti-bacterial, agent. Suitable agents include the antibiotics tetracycline, doxycylcine and ampicillin. Other agents suitable for treating oral infections will be apparent to one skilled in the art.

A method for treating halitosis comprises contacting the oral cavity of a patient that displays symptoms of halitosis with a compound of formula (I), preferably a preparation containing a compound of formula (I) such as a toothpaste, gum, gel or mouthwash formulation. The compound of formula (I) will reduce or remove the halitosis.

Halitosis can be prevented by contacting the oral cavity with a compound of formula (I), preferably a preparation containing a compound of formula (I) such as a toothpaste, gum, gel or mouthwash formulation, prior to the development of offensive odours. The compound of formula (I) will prevent halitosis from occurring in the patient. In the context of a mouthwash, the patient can “gargle” with the mouthwash to ensure all parts of the oral cavity are brought into contact with the active compound.

A kit or pack comprising a compound of formula (I), or a medicament comprising a compound of formula (I), and instructions directing the user to use the compound to treat halitosis, is included in the invention. Preferably, the instructions recite that the compound or medicament is to be used regularly, preferably at least once daily and more preferably every morning and evening, as part of a regular oral health routine.

1. A method for the prevention or treatment of halitosis wherein the method comprises administering to a subject in need of such prevention or treatment, a morpholino compound having the general formula (I) 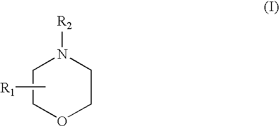
wherein R₁ is a straight or branched alkyl group containing 8 to 16 carbon atoms at the 2- or 3-position of the morpholino ring, and R₂ is a straight or branched alkyl group containing 2 to 10 carbon atoms, substituted with a hydroxy group except in the alpha-position, or a pharmaceutically acceptable salt thereof.

2. The method according to claim 1, wherein the sum of the carbon atoms in the groups R₁ and R₂ of the morpholino compound is at least 10.

3. The method according to claim 1, wherein R₂ of the morpholino compound terminates with the hydroxy group.

4. The method according to claim 1, wherein the morpholino compound is 3-(4-propyl-heptyl)-4-(2-hydroxyethyl) morpholine.

5. The method according to claim 1, wherein the method comprises administering an antimicrobial agent.

6. The method according to claim 1, wherein the morpholino compound is formulated with medicament of a pharmaceutically acceptable excipient.

7. The method according to claim 1, wherein the morpholino acompound is administered as a component of a toothpaste, gum or mouthwash.

8. The method according to claim 1, wherein the morpholino acompound reduces the level of Volatile Sulphur Compounds emitted from the oral cavity.

9. A kit for treating halitosis, comprising a morpholino compound having the general formula (I) 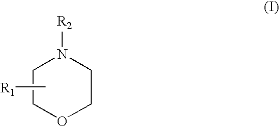
wherein R₁ is a straight or branched alkyl group containing 8 to 16 carbon atoms at the 2- or 3-position of the morpholino ring, and R₂ is a straight or branched alkyl group containing 2 to 10 carbon atoms, substituted with a hydroxy group except in the alpha-position, or a pharmaceutically acceptable salt thereof, or
a medicament containing said compound or salt thereof, wherein said kit further comprises, instructions that the compound or medicament is to be used for the treatment of halitosis.
